(19) United States
(12) Patent Application Publication
(10) Pub. No.: US 2024/0051932 A1
(43) Pub. Date: Feb. 15, 2024

(54) LABILE AND COHERENT REDOX-SILENT ANALOGUES FOR VITAMIN E ENHANCEMENT

(71) Applicant: Hermay Labs Corporation, (US)

(72) Inventors: Leonard Wiebe, Edmonton (CA); Eu Peter, Balwyn North (AU); John Yao, Edmonton (CA)

(21) Appl. No.: 17/761,978

(22) PCT Filed: Oct. 27, 2021
(86) PCT No.: PCT/CA2021/051518

§ 371 Date: May 5, 2022

(51) Int. Cl. C07D 311/72; A61K 51/04; A61K 49/00; A61K 47/54

(52) U.S. Cl. CPC C07D 311/72; A61K 51/0421; A61K 49/0052; A61K 47/543; A61K 47/54

(57) ABSTRACT

The present invention provides for a novel method for the synthesis of Vitamin E analogues and useful derivatives. The invention provides for novel methods for the derivatization of Vitamin E to generate molecules with improved bioavailability, radiolabelling capabilities for diagnostic use, and improved methods for the isolation or purification of Vitamin E from natural plant oil sources.

### FIELD OF THE INVENTION

The present invention pertains to the field of chemical modification of natural products, nutraceuticals and antioxidants, as applied to Vitamin E

### BACKGROUND OF THE INVENTION

All of the publications, patents and patent applications cited within this application are herein incorporated by reference in their entirety to the same extent as if the disclosure of each individual publication, patent application or patent was specifically and individually indicated to be incorporated by reference in its entirety.

The Vitamin E family is comprised of eight chromanol analogues that fall into two sub-families, tocopherols (TPs) and tocotrienols (T-3s) (FIG. 1). These families are further identified and differentiated by the hydrocarbon chain at chromanol-C2. Whereas T-3s have a C-2 isoprenoid side chain with three non-conjugated double bonds (C3′-C4′, C7′-C8′, C11′-C12′), the TPs have an identical but fully saturated chain. The four naturally occurring TP and T-3 analogues (α-, β-, γ-, δ-) differ from each other only through variations in methyl substitutions at C5, C7 and C8 on the aromatic chromanol ring.

Vitamin E analogues are important natural antioxidants that protect cells by interacting with free radicals. There is growing evidence that other Vitamin E medicinal properties including anti-cancer, anti-inflammatory, and neuroprotective activity may be as important as their antioxidant action. Specific biochemical interactions may lower blood cholesterol and blood pressure, reverse atherosclerosis, minimize stroke-related brain damage, stimulate hair regrowth, and prevent sun-damage to skin. The Vitamin E analogue γ-T-3, present in many plant oils but especially palm, annatto and rice bran oils; is of particular interest to cancer researchers.

The pharmacology, metabolism and molecular biology of Vitamin E continue to be the subject of scientific investigation. In general, the bicyclic (chromanol) portion is responsible for the antioxidant properties, whereas the hydrocarbon side-chain at C2 has two functions: the proximal portion imparts signalling activity, and the distal (terminal) hydrocarbon tail imparts additional hydrophobicity which facilitates interaction with lipophilic cell components (FIG. 2). The side-chain terminus is subject to oxidative attack by cytochrome P450 4F2 (CYP4F2) at C-13′ leading to o-hydroxylation followed by a cascade of successive beta oxidations starting with C-13′, destroying the side-chain and leaving the water-soluble carboxyethylhydroxychroman or carboxymethylbutyl hydroxychroman cores. Oxidation of the aromatic ring of the chroman moiety has also been reported to give rise to minor metabolites.

The art is in need of novel analogues of Vitamin E that will support their in vivo, in vitro and in situ detection and quantitation in biological matrices. Further, the art is in need of improved methods for the isolation of Vitamin E, and for improved pro-drugs that will promote their absorption from the intestinal lumen and improve general transport across plasma membranes.

### SUMMARY OF THE INVENTION

The present invention provides for the synthesis of novel derivatives of Vitamin E analogues. These novel Vitamin E adducts include, but are not limited to, compounds which enable pharmacological, pharmaceutical, histopathological and molecular studies in vivo, as well as those that simplify or improve recovery of Vitamin E from its natural sources, and improved oral bioavailability following ingestion by animals or humans.

Further, the present invention provides for producing nutraceutically, pharmacologically and pharmaceutically active compounds, including compounds useful for the diagnosis and treatment of clinical disorders as ascribed to Vitamin E in the literature, as agents useful in discovery of pharmacological and pharmacokinetic characteristics of Vitamin E, as intermediate agents useful in the isolation of Vitamin E from its natural sources, and as aids to improving the oral absorption and bioavailability of natural Vitamin E.

In one aspect the present invention provides for a compound of formula (I)

Vitamin E-LNK-REG (I)

wherein Vitamin E is alpha-tocopherol, beta-tocopherol, gamma-tocopherol, delta-tocopherol, alpha-tocotrienol, beta-tocotrienol, gamma-tocotrienol, or delta-tocotrienol; wherein LNK is a linear or branched hydrocarbon or substituted hydrocarbon linked to the C6 oxygen on the Vitamin E component via an ether, carbamate or ester bond and having a reactive centre suitable for displacement by a nucleophilic reporter element; and wherein REG is a reporter element or group that is a chemical nucleophile. In one embodiment REG is a halide, azide, reporting moiety that acts as a fluorophore, chromophore, a radioactive element, or a nuclear magnetic resonance responsive center. In a further embodiment the halide is iodine, bromine, fluorine, or chlorine. In another embodiment the halide is a radioisotope of is Iodine, Bromine, Fluorine, Chlorine. In another embodiment the nuclear magnetic resonance responsive center is a mono-fluorinated, di-fluorinated, tri-fluorinated or polyfluorinated entity. In another embodiment the fluorophore is nitrobenzoxadiazole, or sulfo-cyanine5 fluor. In another embodiment the chromophore is an activated aromatic. In a further embodiment the activated aromatic is a nitrated aromatic.

In another aspect the present invention provides for a compound of formula (II)

Vitamin E-LNK-GRP (II)

wherein Vitamin E is alpha-tocopherol, beta-tocopherol, gamma-tocopherol, delta-tocopherol, alpha-tocotrienol, beta-tocotrienol, gamma-tocotrienol, or delta-tocotrienol; wherein LNK is a linear or branched hydrocarbon or substituted hydrocarbon linked to the C6 oxygen on the Vitamin E component via an ether, carbamate or ester bond and having a reactive centre suitable for displacement by a nucleophilic reporter element; and wherein GRP is a functional element that modifies a property of the Vitamin E component of the compound. In one embodiment GRP is a mono-saccharide, di-saccharide, poly-saccharide, glyceric acid, amino acid, or inorganic acid; resulting in reduction of the lipophilicity of the Vitamin E component of the compound. In a further embodiment the mono-saccharide, di-saccharide, or poly-saccharide is uronic acid, gluconic acid, glycuronic acid, ascorbic acid, lacturonic acid, or saccharic acid. In another embodiment GRP provides for improved oral absorption in a mammal. In a further embodiment LNK is linked to the C6 oxygen on the Vitamin E component by a di-ester and GRP is a glyceride. In another embodiment GRP provides for improved oral bioavailability in a mammal. In a further embodiment LNK is linked to the C6 oxygen on the Vitamin E component by a di-ester and GRP is a glyceride. In another embodiment LNK is linked to the C6 oxygen on the Vitamin E component by mono esterification with glyceric acid or mono-esterification via a dicarboxylate linker, such that the compound may undergo acid hydrolysis to yield Vitamin E, glycerin and dicarboxylate.

In another aspect, the present invention provides for a method to isolate Vitamin E from natural plant oil distillate containing Vitamin E comprising esterification of hydroxy groups on glucuronic acid sugar by addition of trifluoroacetate generating protected glucuronic acid, addition of the protected glucuronic acid to said natural plant oil distillate generating Vitamin E glucoronate, addition of dilute fluoroacetate to said Vitamin E glucoronate and extracting Vitamin E from the resulting mixture with water. In one embodiment extracting Vitamin E from the resulting mixture with water further comprises addition of diethyl ether and dilute aqueous sodium carbonate.

In another aspect, the present invention provides for a compound of formula (III)

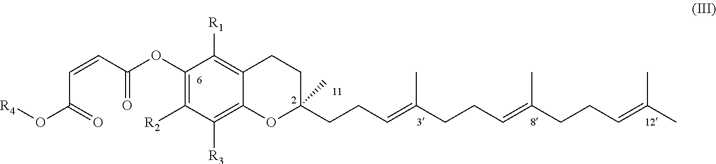

Wherein R₁ is H, or CH₃; R₂ is H or CH₃; R₃ is H or CH₃; and R₄ is H, Vitamin C, or H₂NC(CH₂OH)₃.

In another aspect, the present invention provides for a compound of formula (IV)

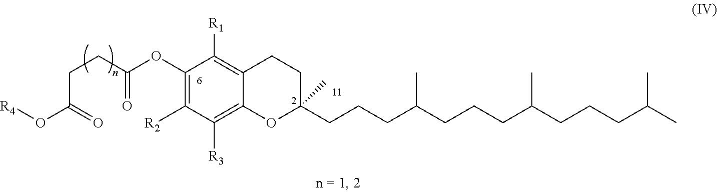

Wherein R₁ is H, or CH₃; R₂ is H or CH₃; R₃ is H or CH₃; and R₄ is H, Vitamin C, or H₂NC(CH₂OH)₃.

### BRIEF DESCRIPTION OF THE FIGURES

FIG. 1 shows a schematic of the chemical structures of the eight natural analogues of Vitamin E;

FIG. 2 shows the gamma tocotrienol (γ-T-3) chemical structure and general molecular biology of its major structural domains;

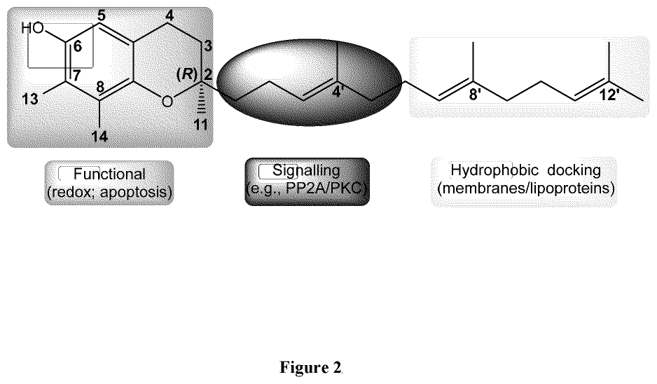

FIG. 3 shows a schematic of the generalized method of synthesis of compounds of the present invention;

FIG. 4 shows a schematic of the generalized method of synthesis of F-γ-T-3 and [¹⁸F]F-γ-T-3 using a tosylate and mesyl and with F/[¹⁸F] as the reporting element/group (REG);

FIG. 5 shows a representative HPLC radio-uv co-chromatogram of the F-γ-T-3 and TsO-γ-T-3 reaction mixture after radiofluorination as synthesized from γ-T-3;

FIG. 6 shows the partial ¹H NMR spectrum of F-γ-T-3;

FIG. 7 shows a Positron Emission Tomographic (PET) image of F-18 biodistribution in a mouse following i.v. tail vein injection of [¹⁸F]F-γ-T-3;

FIG. 8 shows a schematic of generalized methods to synthesize hydrophilic Vitamin E derivatives;

FIG. 9 shows a schematic of the synthesis of a Vitamin E ester designed for transport across plasma membranes;

FIG. 10 shows schematic reactions for ‘click’ insertion of reporting elements (REs) on azide- and alkyne-derivatized Vitamin E;

FIG. 11 shows chemical structures of compounds created using the methods of the present invention;

FIG. 12 shows the general copper catalyzed “click” reaction involving alkynes and azides;

FIG. 13 shows a reaction schematic of the preparation of NBD-APy-T-3; and

FIG. 14 shows further exemplary compounds capable of production using the methods of the present invention.

### DETAILED DESCRIPTION OF THE PRESENT INVENTIONS

The present invention provides, in part, for the preparation of a radiolabelled tracer for use in in vivo studies of Vitamin E, including but not limited to radiolabelled gamma tocotrienol (γ-T-3).

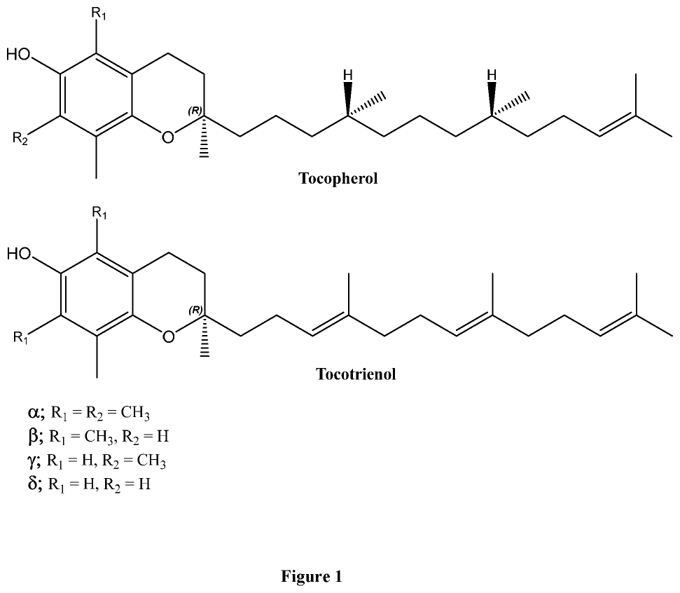

As used herein, “Vitamin E” means all 8 natural analogues of the vitamin E family and is used as a collective name for all eight natural analogues of this chromanol family (4 tocopherols and 4 tocotrienols); all as further described in accordance with IUPAC/IUB terminology (1982, Eur J Biochem 123:473-475) and presented in FIG. 1. The abbreviations for tocopherol (TP) and tocotrienol (T-3) are those provided by 1973 Recommendations of the IUPAC-IUB Commission on Biochemical Nomenclature (CBN), Nomenclature of Quinones with Isoprenoid Side-Chains, (1975, Eur. J. Biochem. 53:15-18).

As used herein, “Alkyl” refers to straight or branched chain alkyl groups having between 1-12 carbon atoms, most commonly 1-4 carbon atoms. Alkyls may be substituted or unsubstituted, cycloalkyl or short alkyl groups bearing one or more substituents such as hydroxy, alkoxy, aryl, mercapto, halogen, trifluoromethyl, cyano, nitro, amino, carboxyl, carbamate, sulfonyl, sulfonamide, and others. Examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, cyclohexyl, and others.

As used herein, “Alkoxy” refers to a compound of the formula RO-, where R is alkyl (which may be substituted or unsubstituted unless specified otherwise) as given above.

As used herein, “Alkenyl” refers to straight or branched chain hydrocarbyl groups such as alkyl as described above (including substitution) and having at least one carbon-carbon double bond.

As used herein “Alkynyl” refers to straight or branched chain hydrocarbyl groups such as alkyl, substituted and unsubstituted, saturated and unsaturated and having at least one carbon-carbon triple bond.

As used herein “Aryl,” refers to a monocyclic carbocyclic ring system or a bicyclic carbocyclic fused ring system having one or more aromatic rings, including but not limited to naphthyl, phenyl, tetrahydronaphthyl and others. Aryl groups may be substituted or unsubstituted, and when substituted can be substituted with 1, 2, 3, 4, or 5 substituents selected from a wide range of substituents such as alkyl, alkenyl, alkenyloxy, alkoxy, alkoxyalkoxy, alkoxycarbonyl, alkylcarbonyl, alkylsulfonyl, alkylthio, alkynyl, aryl, aryloxy, azido, arylalkoxy, arylalkyl, aryloxy, carboxy, cyano, formyl, halogen, haloalkyl, haloalkoxy, hydroxy, hydroxyalkyl, mercapto, nitro, sulfonate.

As used herein “Halo” refers to an atom selected from fluorine, chlorine, bromine and iodine.

As used herein “LEG” means a leaving component which includes nucleophilic species that accept a pair of electrons from a nucleophile (an electron donor, herein called the reporter element/group, REG). LEGs include but are not limited to halides (e.g., I, Br, Cl), sulfonic acids (e.g., tosyl, nosyl, mesyl), carboxylic acids and protonated amines.

As used herein “REG” means reporter elements/groups that are chemical nucleophiles that can displace an LEG through nucleophilic reaction. Included are halides (including radioisotopes of I, Br, F, Cl), azides, reporting moieties (REs) that act as fluorophores (including but not limited to NBD, Cy5 and other common fluorescent reagents), chromophores (including but not limited to nitrated aromatics and other activated aromatics), radioactive (including but not limited to F-18, F-19, I-123, I-124, I-125, I-131) and NMR-responsive centers (including mono-, di-, tri- and polyfluorinated entities).

As used herein “CG” means Click groups, which include reactive components that are useful for introduction of reporter elements (CCR) by reacting via ‘click’ chemistry (see, for example, Angew. Chem. Int. Ed., 2002, 41:2596). The CG (azide or ‘yne’ carbon triple bond) on the LNK, reacts with the respective ‘yne’ or azide (CCR) to form the desired Vit E-CCR (Equation 1).

Vit E-LNK-CG′+CCR(‘yne’ or N₃, respectively)→Vit E-LNK-CCR (Equation 1)

As used herein “CCR” means click chemistry reporters, being reporting moieties including fluorophores (including but not limited to NBD, Cy5 and other common fluorescent reagents), chromophores (including but not limited to nitrated aromatics and other activated aromatics), radioisotopes (including but not limited to F-19, I-123, I-124, I-125, I-131) and NMR-responsive centers (including mono-, di-, tri- and polyfluorinated entities). These CCRs are distinguished by having either azide or ‘yne’ foci that will react with ‘yne’ or azide on the Vit E-LNK-N3/‘yne’ synthon.

As used herein “nucleophiles” means chemical species that donate an electron pair to form a chemical bond in relation to a reaction. Examples include, but are not limited to, halides, thiols, azides, amines and nitriles.

As used herein “LNK” means a linear or branched hydrocarbon or substituted hydrocarbon (C=1-12)(for example, alkyl, alkenyl, alkynyl, aryl) linked to C6-O— via an ether, carbamate or ester bond and having a reactive centre (leaving element/group, LEG, for example, sulfonyl, azide, halide) suitable for displacement by a nucleophilic reporter element (RE; for example, halogen; radiohalogen; flurophore, NMR reporter, chromophore).

The present invention provides for novel methods for the incorporation of reporting elements/groups (REGs) into the Vitamin E structure as generally described as REG-LNK-Vitamin E. The novel compounds produced by the methods of the present invention have particular utility in the non-invasive imaging, spatial and kinetic analyses of Vitamin E distribution in cells, issues and whole organisms and provide for greater accessibility, sensitivity, and specificity in that imaging; without destruction of the innate properties of Vitamin E analogues. One skilled in the art will recognize the utility of the use of known Reporting Elements/Groups of the REG-LNK-Vitamin E structure described herein, for whole-body imaging (radioactive—PET, SPECT and planar; optical—fluorimetry; magnetic resonance—MRI, MRS), histopathology, and molecular analysis, for applications in pharmacology, pharmacokinetics, drug metabolism and molecular biology the treatment of disease.

The present invention further provides for novel methods for the recovery of Vitamin E from natural sources, including but not limited to, plant oils. Whereas current methods require distillation and chromatographic sequences, the use of in situ ester derivatization at C6-O— on Vitamin E converts the highly lipophilic Vitamin E to more hydrophilic adducts that can be removed from the original oil by aqueous extraction and recovered by simple aqueous extraction of water-soluble moieties and leaving behind the original concentrated lipophilic Vitamin E analogues. The present invention provides for the use of polyhydric acids as said hydrophilic adducts, including but not limited to glucuronic acid, ascorbic acid, sugar acids and natural and synthetic polyhydroxylated monomers, dimers and polymers including suitable sugar derivatives.

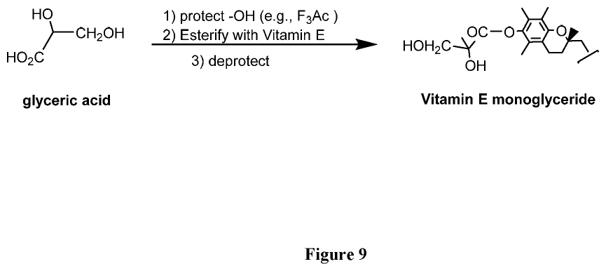

The present invention also provides for the novel method for synthesis of the Vitamin E-mono-glyceric acid ester and its application for use as an enhancer of Vitamin E bioavailability. As shown by schematic in FIG. 9, by derivatization of Vitamin E through esterification with glyceric acid at C6-O, the resulting monoglyceride adduct becomes a structural mimic of natural lipids and thus utilizes the body's natural method for moving lipids across membranes (e.g., intestinal epithelium; plasma membranes in general). Furthermore, by derivatization of Vitamin E through esterification with polyhydric acids (e.g., gluronate) at C6-O, greater dispersion of Vitamin E can be attained through self-association, micelle formation and emulsification in the intestinal lumen.

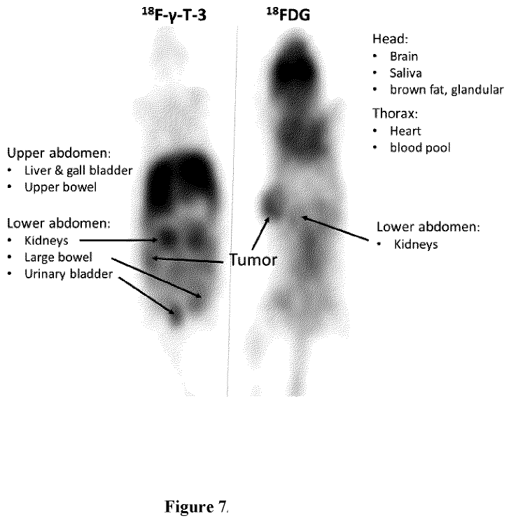

As known in the prior art, there are very few options for the introduction into Vitamin E analogues of reporter elements/groups (REGs), such as radioisotopes, fluorescent species, and NMR-responsive perfluoro moieties: the chemical structure of Vitamin E and their ascribed regional structure-activity relationships are simply too constrained. The radiolabeling options fall into two main categories: those that impact the redox potential and bio-oxidation properties (e.g., alteration/substitution at C6-OH), and those that alter signalling, docking and metabolic degradation ascribed to the C2 chain (e.g., substitution on isoprenoid double bonds of T-3s). The latter option does not apply to TPs, and for T-3s, requires creation of a reactive centre somewhere along the chain; this requires substantial chemical synthesis and creates critical alterations in the biophysical properties of these molecules. Incorporation of tracer elements at aromatic sites other that C6-O would similarly necessitate extensive chemical modification, a tedious and difficult exercise resulting in compounds with modulated interaction with membranes and macromolecular targets, as well as altered absorption and biodistribution patterns. Many of the reported synthetic derivatives of the natural TPs and T-3s are based on substitution on C6-OH. The present invention provides for the novel introduction of REGs (radiolabels, fluorescent species, fluorinated components) via a prosthetic linking arm at C6-O as a useful methodology for reporter-labeling of all Vitamin E analogues. FIG. 7 presents F-18 PET images showing the biodistribution of radioactivity in a mouse following intravenous tail veil injections of a compound of the present invention, [18F]F-γ-T-3 (left image) and reference [18F] fluoro-D-glucose (FDG, right image). The [18F]F-γ-T-3 image was captured 2 hours after injection, and the [18F] fluoro-D-glucose was obtained 90 minutes post-injection, using the same animal 24 hours following the [18F]F-γ-T-3 study.

The present invention contemplates various composition of linking structures LNK; by way of non-limiting example alkyl 1C to 12C; with the chemical options of the LNK at C6-O (e.g., O-alky; O-carbamate) and reaction conditions obvious to one skilled in the art. Those skilled in the art will recognize that chemical structures of the products are dependent upon the particular starting compound (Vitamin E analogue) and on the functional ingredients (e.g., LNKs, LEGs, REGs, catalysts, initiators); and that the reaction conditions disclosed herein may not be optimized for yield or purity of particular starting compounds and functional ingredients unless explicitly stated as such. The masses of the reagents, reaction times and temperatures utilized in the synthesis of compounds arising from the use of the methods described herein will fall with ranges normally reported for other (non-Vitamin E) compounds. ‘Click’ reactions (as further described herein) are designed to work with C6-O-alkylazido- or C6-O-alkynyl intermediates (LNKs) and their counter reagents (alkynyl-functional moiety and azido-functional moiety, respectively).

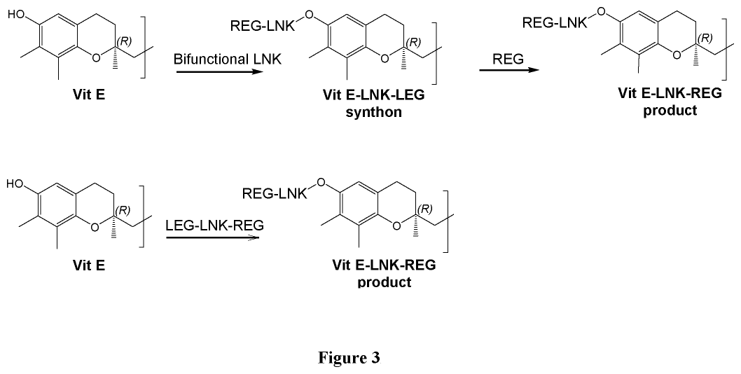

The present invention provides for, but is not limited to, a method of nucleophilic displacement of an appropriate LEG bonded attached to Vitamin E at C6-O via a LNK moiety. This nucleophilic displacement of LEG is effected by REG, thereby introducing an REG into the structure. In the case of radiofluorination to make [¹⁸F]F-Vitamin E, the displacement of a LEG on the appropriate Vitamin E synthon by a nucleophilic REG (FIG. 3 and Equation 2). The products obtained from the general process disclosed in FIG. 3, are particularly useful in the preparation of Vitamin E analogues with improved bioavailability, solubility, non-invasive imaging, and providing useful intermediates in the

Vitamin E-LNK-LEG+REG→Vitamin E-LNK-REG+LEG. (Equation 2)

The present invention provides for, a novel method of introducing nucleophilic REGs into Vitamin E using bifunctional LNKs, as known in the art and by way of non-limiting example ¹⁸F(CH₂)ₙX (n=1-3, X=Br, OMes, OTos) (1988, J Label Compd Radiopharmaceut. 25:201-216). This synthesis is achieved by first linking the REG to the LNK, then connecting LNK to Vitamin E at C6-O (Equation 3). For example, radiofluorinate and mesylate propanol to make 1-mesyl-3-fluoropropane and then connect this to T-3 to make [¹⁸F]F-γ-T-3.

Vitamin E+LEG-LNK-REG→Vitamin E-LNK-REG+LEG (Equation 3)

One skilled in the art will recognize that the synthesis strategy provided for in Equation 2 and Equation 3 are capable of producing the same product, and both methods are contemplated to produce compounds of the present invention, with the election of synthesis strategy based upon particular needs and reaction conditions.

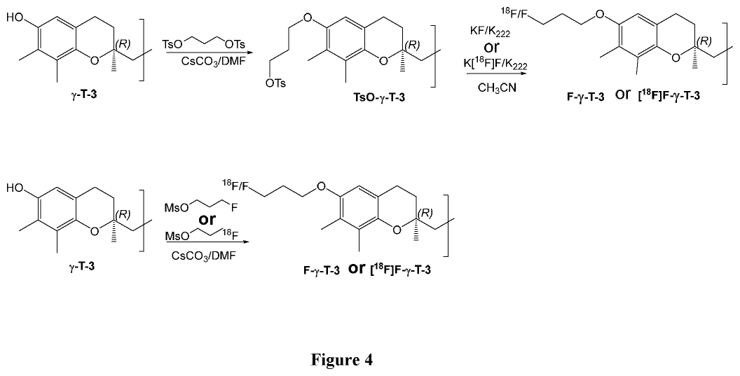

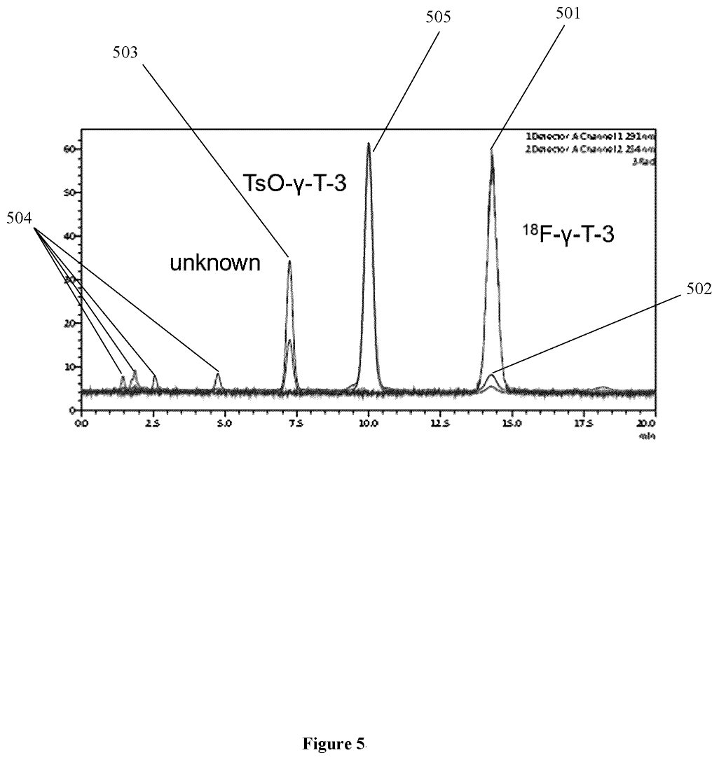

The present invention provides for, but is not limited to, a novel method of introducing nucleophilic REGs into Vitamin E using bifunctional LNK structures, as known in the art. By way of non-limiting example, FIG. 3 provides a general schematic of the reactions, and FIG. 4 provides for mesylation of 3-fluoro-1-propanol followed by nucleophilic substitution of its mesyl group with γ-T-3 to furnish the target F-γ-T-3 in 61% yield (reagents and conditions: (a) MsCl, Et₃N, DCM, room temperature (r.t.), 30 min, 92.7%; (b) CsCO₃, DMF, r.t.), and radiofluorination of TsO-γ-T-3 provided adequate radiochemical yield (RCY 12%) without further optimization. HPLC afforded 0.5-1 GBq high purity (RCP>99%) [¹⁸F]F-γ-T-3. A specific gradient HPLC method produced high radiochromatographic purity product with no detectable radiochemical impurities. The high specific activity product obtained via nucleophilic radiofluorination is not detectable by ultraviolet light absorption, but addition of internal standard F-γ-T-3 produced a peak at 14.4 min, directly underneath the radioactive peak of [¹⁸F]F-γ-T-3 upon co-chromatography (FIG. 5). As shown in FIG. 5, the 291 nm and 254 nm peaks eluting at 14.4 min, and the radioactive peak (14.4 min; trace 501) represent the co-mixture of authentic F-γ-T-3 (trace 502) and product [¹⁸F]F-γ-T-3 in the reaction mixture. Absorption peaks in trace 503 (291 nm) correspond to the major uv absorption band of the methyl-substituted 6-chromanol (benzopyran) ring system (e.g., F-γ-T-3, TsO-γ-T-3), and the trace 504 (254 nm) is a less selective indicator of aromaticity. The main uv absorption peaks, 505, eluting at 10.1 min (291 nm and 254 nm) represent unconsumed TsO-γ-T-3; the absorption peaks eluting at earlier times are unidentified. Standard workup of these compounds requires attention to their potential instability under silica gel chromatography and sensitivity to ultraviolet light.

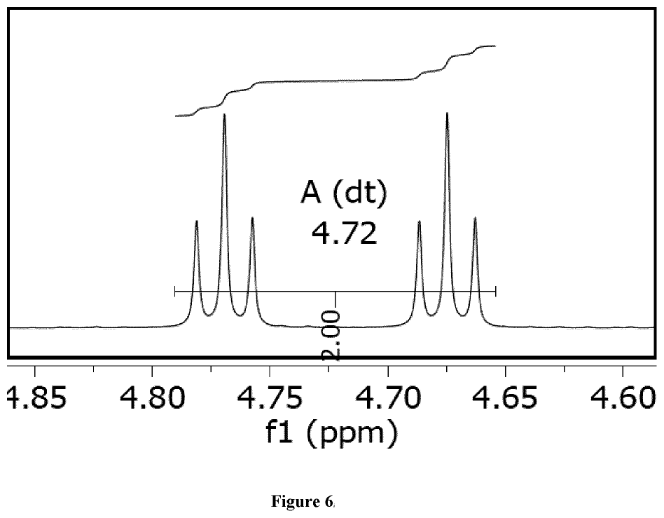

By way of non-limiting example, F-γ-T-3 and TsO-γ-T-3 were synthesized from γ-T-3 in acceptable chemical yields of 61% and 48%, respectively (FIG. 4). The isocratic HPLC system developed for this work provided good separation between TsO-γ-T-3 and F-γ-T-3. A chromatogram of a mixture of TsO-γ-T-3 and F-γ-T-3 showed the presence of several minor impurities which were not identified (FIG. 5). FIG. 6 shows the partial ¹H NMR spectrum of F-γ-T-3 which reveals the distinctive coupling pattern of the fluoropropyloxy substituent at C6-O; especially CH₂-1 of F-γ-T-3 which showed the distinctive coupling pattern of dt (doublet of triplet) with coupling constants J=47.1 and 5.9 Hz, indicating F-H and H-H coupling, respectively. These resonances are well-separated from other aliphatic-H resonances and are therefore useful in distinguishing F-γ-T-3 from non-substituted T-3s.

By way of non-limiting example, [¹⁸F]F-γ-T-3 was produced and used at no-carrier-added (NCA) specific activity (SA). The theoretical SA of NCA [¹⁸F]fluoride is 6.3 TBq/μmol, but in reality, unintentional addition of fluorine through the ubiquitous presence of fluorine in reagents and materials reduces this to 30-150 GBq (1-5 Ci/μmol) of NCA product, although SA's of 0.1-1.9 TBq (3-51 Ci)/μmol have been reported (J Nucl Med. 2012, 53:434). At 150 GBq/mmol, an injected radioactivity dose of 14.8 MBq represents approximately 1 nmol of F-γ-T-3, an amount not likely to modulate most Vitamin E-related processes and in line with the low Vitamin E concentrations found in tissues. Because pharmacokinetic parameters of Vitamin E analogues, in humans at least, do not appear to be dose dependent over a large dose range, specific activity may not be critical to the effectiveness of labeled Vitamin E analogues as diagnostic agents.

### Example 1: General Conditions for Preparation

For the preparation, analysis and quantification of compounds presented in the Examples, the following general conditions were applied. Those skilled in the art will recognize that these are general reaction conditions and procedures, capable of variation as known in the art.

Solvents for reactions were purified by successive passage through columns of alumina and copper under an argon atmosphere. Reagents were purchased from commercial sources and used without further purification unless noted otherwise. All reactions were carried out under a positive-pressure argon atmosphere and monitored by thin layer chromatography (TLC) on Whatman MK6F silica gel micro TLC plates (25 μm thickness) or Silica Gel G-25 UV254 (0.25 mm) microplates using hexanes:EtOAc (1:3, v/v) (solvent system A) and hexanes:EtOAc (1:1, v/v) (solvent system B) as developing solvents. TLC spots were detected under ultraviolet light (uv) and/or by charring with a solution of anisaldehyde in ethanol, acetic acid and H₂SO₄. Column chromatography was carried out on Merck 7734 silica gel (100-200 μm particle size).

For analytics, ¹H and ¹³C NMR spectra were recorded at 498.118 MHz and 125.266 MHz, respectively, and ¹⁹F NMR spectra were recorded at 468.652 MHz. ¹H/¹⁹F NMR chemical shifts are referenced to TMS (0.0, CDCl₃) and ¹³C NMR chemical shifts are referenced to CDCl₃ (d 77.23). ¹H NMR data are reported as though they are first order and peak assignments are based on 2D-NMR (¹H-¹H COSY and HMQC) experiments (FIG. 6). Mass spectra were recorded on either an Agilent 1100 LC/MS using an Agilent Zorbax C-18 column (2.1×50 mm, 5 μM) or Q Exactive™ Hybrid Quadrupole-Orbitrap™ Mass Spectrometer with Xcalibur Data Acquisition and Interpretation Software.

For radiofluorination, acetonitrile (CH₃CN) and Kryptofix2.2.2 (K₂₂₂) were obtained from Merck (Darmstadt, Germany), and dry dimethyl sulfoxide (DMSO) was purchased from Sigma Aldrich. Sep-Pak light, Accell Plus QMA and Alumina N cartridges were from Waters, USA. Phenomenex Luna pre-column (C18/2, 50×10 mm; 5 μm), Phenomenex Nucleosil columns (C18, 250×10 mm; 5 μm and C18, 250×4.6 mm) and 0.22 μm Millex GS and LX filters were from Millipore, USA. NCA [¹⁸F]fluoride was obtained from a PETtrace 16.5 MeV cyclotron incorporating a high-pressure niobium target (Cyclotek(AUST) Pty. Ltd.) via the ¹⁸O(p,n)¹⁸F nuclear reaction. F-18 Separation cartridges (Waters Accell Plus QMA Sep-Pak Light, Kent, UK) were pre-conditioned with 0.5M K₂CO₃ and subsequently rinsed with water. Radio-HPLC analyses were performed using a Shimadzu HPLC (SCL-10AVP system controller, SIL-10ADVP auto injector, LC-10ATVP solvent delivery unit, CV-10AL control valve, DGU-14A degasser, and SPD-1OAVPV detector, MD, USA) Q6 coupled to a scintillation detector (Ortec 276 Photomultiplier Base with Preamplifier, Ortec 925-SCINT ACE mate Preamplifier, Amplifier, BIAS supply and SCA, and a Bicron 1M 11/2 Photomultiplier Tube).

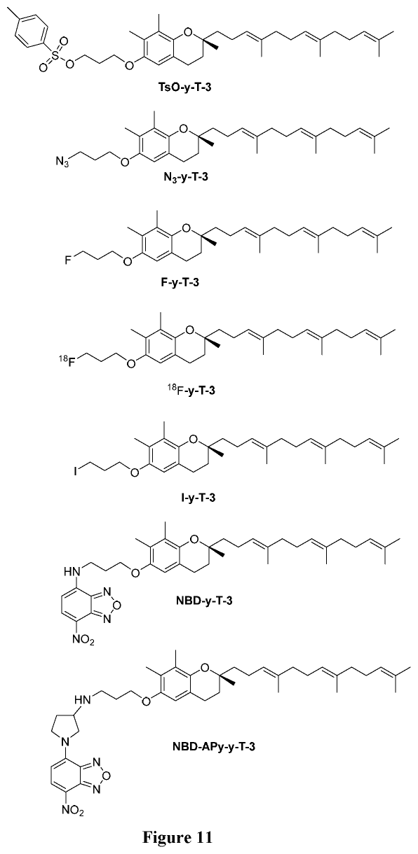

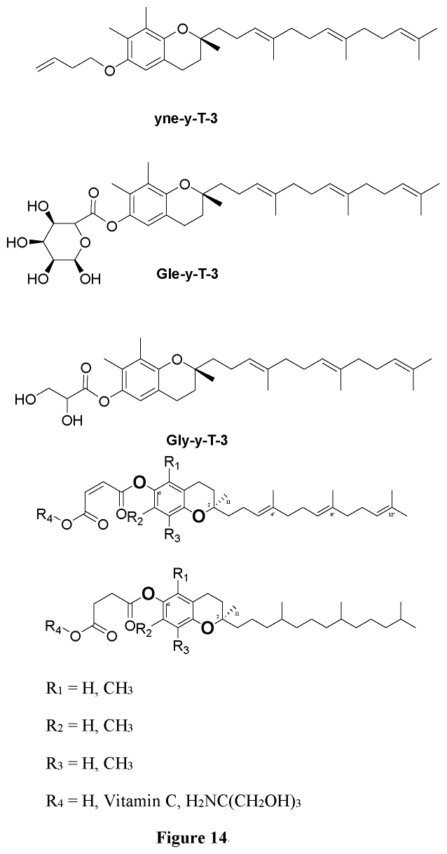

FIG. 11 provides example compounds made using the methods described herein and using those methods and conditions provided for in the disclosed examples; while FIG. 14 provides additional compounds producible by use of the methods described herein.

### Example 2: (R)-6-(3-Fluoropropoxy)-2,7,8-trimethyl-2-(4,8,12-trimethyltrideca-3,7,11-trien-1-yl)-chromane (F-γ-T-3)

Powdered Cs₂CO₃ (3.18 g, 9.77 mmol) was added to a mixture of 3-fluoropropyl mesylate (1.52 g, 9.77 mmol) and γ-T-3 (1.50 g, 3.66 mmol) in DMF (15 mL). The resulting mixture was stirred at r.t. overnight, and then diluted with Et₂O (100 mL) and washed with H₂O (50 mL). The aqueous solution was extracted with Et₂O (2×50 mL) and the resulting organic solution was washed with brine (50 mL), dried over anhydrous Na₂SO₄ and filtered. The filtrate was concentrated and purified by silica gel column chromatography eluted with 0-5% EtOAc in hexane to afford F-γ-T-3 as a yellowish oil (1.05 g, 61%): ¹⁹F NMR (468.652 MHz, CDCl₃): δ=−11.71 (tt, J=47.1, 25.5 Hz); ¹H NMR (498.118 MHz, CDCl₃) δ=6.50 (s, 1H, Ar), 5.27-5.12 (m, 3H), 4.72 (dt, J=47.1, 5.9 Hz, 2H, CH₂-1), 4.06 (t, J=6.0 Hz, 2H, CH₂-3), 2.85-2.72 (m, 2H), 2.28-2.17 (m, 10H, including CH₂-2), 2.17-2.10 (m, 4H), 2.05 (dd, J=8.6, 4.9 Hz, 4H), 1.83 (ddt, J=36.5, 13.2, 6.8 Hz, 2H), 1.75 (d, J=1.4 Hz, 3H), 1.71 (dd, J=9.1, 7.6 Hz, 1H), 1.69-1.64 (m, 9H), 1.64-1.59 (m, 1H), 1.33 (s, 3H); ¹³C NMR (125.266 MHz, CDCl3) δ=149.82, 145.95, 135.07, 134.95, 131.21, 125.98, 124.62, 124.26, 117.51, 109.97, 81.74, 80.43, 77.33, 77.08, 76.82, 75.27, 64.57, 64.52, 39.89, 39.77, 39.75, 31.53, 30.92, 30.76, 26.82, 26.66, 25.73, 24.06, 22.66, 22.28, 17.72, 16.04, 15.93, 11.91, 11.89; HRMS (ESI): m/z calculated for C₃₁H₄₈FO₂: 471.3638 [M+H]+; found: 471.3630.

### Example 3: (R)-6-(3-Iodopropoxy)-2,7,8-trimethyl-2-(4,8,12-trimethyltrideca-3,7,11-trien-1-yl)-chromane (I-γ-T-3)

γ-T-3 (0.30 g) was added to a mixture of 1,3-diiodopropane (2 eq) and Cs₂CO₃ (2 eq) 9.77 in DMF (15 mL); this was stirred at r.t. overnight, then diluted with Et₂O (100 mL) and washed with H₂O (50 mL). The aqueous solution was extracted with Et₂O (2×50 mL) and the resulting organic solution was washed with brine (50 mL), dried over anhydrous Na₂SO₄ and filtered. The filtrate was concentrated and purified by silica gel column chromatography eluted with 0-5% EtOAc in hexane to afford F-γ-T-3 as an impure yellowish oil (83%). Repurification by silica gel column chromatography with 0˜10% EtOAc/hexane afforded (I-γ-T-3); 162 mg; Mass 579.5 [M+H]+, calcd 578.26 for C₃₁H₄₇IO₂.

### Example 4: (R)-6-(3-Tosyl-propoxy)-2,7,8-trimethyl-2-(4,8,12-trimethyl-trideca-3,7,11-trien-1-yl) chroman (TsO-γ-T-3)

Powdered Cs₂CO₃ (4.24 g, 13.0 mmol) was added to a mixture of γ-T-3 (2 g, 4.87 mmol) and 1,3-ditosylpropane (5 g, 13.0 mmol) in DMF (20 mL). The resulting mixture was stirred at r.t. overnight. The mixture was then diluted with EtOAc (20 mL) and water (50 mL) and finally extracted with EtOAc (2×20 mL). The combined organic solution was washed with H₂O (50 mL). The organic solution was dried over Na₂SO₄. After filtration, the filtrate was concentrated and purified by silica gel column chromatography eluted with DCM to afford TsO-γ-T-3 as a yellow oil in 48% yield (1.45 g): ¹H NMR (498.118 MHz, CDCl₃): δ=7.78 (d, J=8.2 Hz, 2H, Ar), 7.27 (d, J=7.8 Hz, 2H, Ar), 6.34 (s, 1H, Ar), 5.40-4.97 (m, 2H), 4.28 (t, J=6.1 Hz, 2H, CH₂-1), 3.89 (t, J=5.8 Hz, 2H, CH₂-3), 2.78-2.66 (m, 2H), 2.41 (s, 3H, CH₃), 2.20-2.07 (m, 6H, including CH₂-2), 2.02-1.94 (m, 5H), 1.87-1.49 (m, 18H), 1.35-1.17 (m, 6H), 1.09-0.83 (m, 3H); ¹³C NMR (125.266 MHz, CDCl3) δ=182.23, 149.44, 145.89, 144.65, 132.92, 129.76, 127.85, 125.84, 124.30, 117.42, 109.56, 77.31, 77.06, 76.80, 75.27, 71.42, 67.48, 63.88, 39.88, 39.72, 36.93, 35.00, 31.48, 29.22, 25.71, 24.07, 23.09, 22.63, 22.23, 21.59, 17.70, 16.55, 15.82, 11.88, 11.74; HRMS (ESI): m/z calculated for C38H₅₄₀₅S: 623.3765 [M+H]+; found: 623.3760.

### Example 5: Radiosynthesis of (R)-6-(3-[¹⁸]Fluoropropoxy)-2,7,8-trimethyl-2-(4,8,12-trimethyltrideca-3,7,11-trien-1-yl)-chromane ([¹⁸F]F-γ-T-3)

[¹⁸F]Fluoride in H₂[¹⁸O]O was transferred to the Tracerlab FXFN radiosynthesis module and passed through a pre-conditioned QMA cartridge. Trapped [¹⁸F]fluoride (3-7 GBq) was eluted to the reactor with a solution consisting of K₂C₂O₄ (2.5 mg), K₂₂₂ (10 mg) and K₂CO₃ (10 mL of 5 mg/mL solution) in CH₃CN and H₂O (1 mL, 80:20). This solution was evaporated to dryness at 65° C. under helium flow and vacuum for 7 minutes followed by heating at 120° C. under vacuum for a further 7 minutes. Tosylate precursor (TsO-γ-T-3; 10 mg) in CH₃CN was added to the anhydrous K[¹⁸F]F/K₂₂₂ residue, followed by heating at 100° C. for 10 minutes. The radioactive reaction mixture was then diluted with mobile phase (EtOH-H₂O, 1.5 mL) and transferred to the loop injection vial. The reaction vial was washed further with mobile phase (1.5 mL) and transferred to the loop injection vial. Preparative HPLC (FIG. 4)[(Nucleosil C18, 300 mm×16 mm), mobile phase H₂O:EtOH (90:10), flow rate 3 mL/min] afforded [¹⁸F]F-γ-T-3 (0.5-1.0 GBq; 12% RCY; >99% RCP), in a total preparation time of 45 min. [¹⁸F]F-γ-T-3 was formulated in EtOH:propylene glycol (PPG):saline(0.9%) (25:25:50) and used directly for small animal imaging studies.

### Example 6: (R)-6-(3-azido-propoxy))-2,7,8-trimethyl-2-(4,8,12-trimethyltri-deca-3,7,11-trien-1-yl)-chromane (N₃-T-3)

Cs₂CO₃ (3.18 g, 9.77 mmol) was added to a mixture of 3-azidopropylmesylate (1.75 g, 9.77 mmol) and γ-T-3 (1.50 g, 3.66 mmol) in DMF (15 mL). The resulting mixture was stirred at r.t. overnight. The mixture was diluted with 100 mL of Et₂O and washed with water (50 mL). The aqueous solution was extracted with Et₂O (2×50 mL). The combined organic solution was washed with brine (50 mL). The organic solution was dried over Na₂SO₄. After filtration, the filtrate was concentrated and purified by silica gel column chromatography eluted with 0-5% EtOAc in hexane to afford N₃-T-3 as a yellow oil in 67.8% yield (1.22 g).

### Example 7: (R)-6-(3-(4-amino-NBD-propoxy))-2,7,8-trimethyl-2-(4,8,12-trimethyltrideca-3,7,11-trien-1-yl)-chromane (NBDA-T-3)

Ph₃P (210 mg, 0.768 mmol) was added to a solution of 4-amino-(3-hydroxypropyl)-NBD (64.5 mg, 0.256 mmol) and γ-T-3 (100 mg, 0.244 mmol) in THF (5 mL), followed by the addition of DIAD (156 mg, 0.768 mmol). The resulting mixture was stirred at r.t. overnight. After removal of solvent, the residue was purified by column chromatography eluted with 0-5% EtOAC in DCM to afford NBDA-γ-T-3 as an orange solid in 12% yield (20 mg). ¹H NMR and LC-MS (m/z 645.4).

### Example 8: (R)-6-(3-(4-aminopyrolidin-1-yl-NBD)-propoxy))-2,7,8-trimethyl-2-(4,8,12-trimethyltrideca-3,7,11-trien-1-yl)-chromane (NBD-APy-T-3)

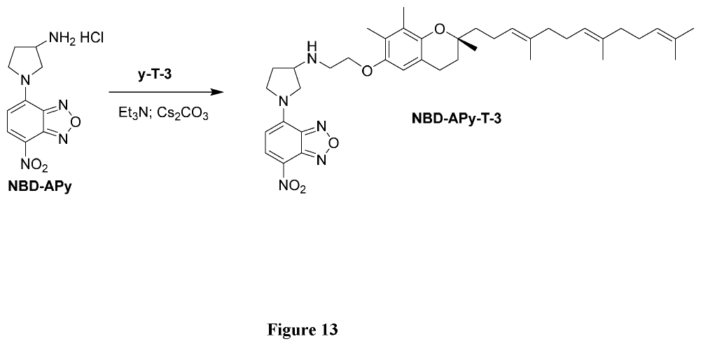

FIG. 13 shows a reaction schematic of the preparation of NBD-APy-T-3. TsO-γ-T-3 (0.15 g, 0.25 mmol) in DMF (1 mL) was added to a mixture of NBD-APy (0.57 mmol) and Et₃N (0.1 mL, 0.741 mmol) in DMF (4 mL). The resulting mixture was stirred at r.t. for 3 hrs. Then, Cs₂CO₃ (0.24 g, 0.74 mmol) was added and stirred at r.t. for 16 hrs. The mixture was quenched with water (20 mL) and extracted with Et₂O (3×25 mL) and EtOAc (2×25 mL). The aqueous solution was concentrated and combined with organic solution, then dried over Na₂SO₄. After filtration, the filtrate was concentrated and purified by silica gel column chromatography eluted with 0-50% EtOAc in hexane to afford NBD-APy-T-3 as an orange semi-oil in 8.9% yield (15 mg).

### Example 9: Hydrophilic Vitamin E Esters

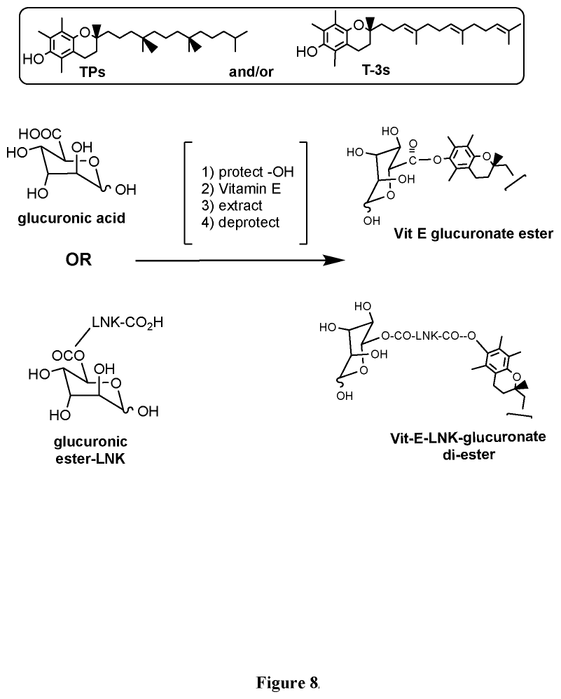

Palm oil distillate is a mixture of moderate molecular weight, highly lipophilic compounds; a typical assay could be TPs 9-13%; T-3s 38-4²%; carotenes <1%; sterols 2-4%; squalene 7-10%; tri-oleins 30-45%. The hydroxy groups of glucuronic acid sugar are esterified with trifluoroacetic anhydride, then the Vitamin E (γ-T-3) in the distillate is esterified by the protected glucuronic acid, to form the Vitamin E glucuronate. This ester is deprotected in situ with a dilute fluoroacetate, and the Vitamin E-glucuronate is extracted from the oily mixture with water. FIG. 8 demonstrates that addition of diethyl ether and work up with dilute aqueous sodium carbonate results in a bilayer, the organic phase containing the Vitamin E extracted from the oil distillate and the aqueous phase containing sodium fluoroacetate and other water soluble components.

FIG. 8 further shows an alternate extraction procedure in which the Vitamin E is linked in situ in the distillate to a sugar via a bifunctional linker (maleic acid). The recovery of Vitamin E follows a similar sequence of extraction and deprotection to afford the original Vitamin E.

### Example 10 Absorption Enhancement Formulation of Vitamin E

Intestinal absorption of Vitamin E is effected through complex biomolecular mechanisms involving intracellular trafficking proteins, nuclear receptor modulation and ATP binding cassette transporters, in addition to its biophysical dispersion in the intestinal lumen. Dispersion is associated with micelle and emulsion formation, which results through the self-assembling properties of Vitamin E (an amphophile). The lipophilic part of Vitamin E consists of a long saturated (TP) or unsaturated (T-3) hydrocarbon chain and a non-ionic slightly hydrophilic hydroxy head, which enables them to reduce interfacial tension (surfactant property) by associating to form micelles which play important roles as emulsifiers and dispersants required for absorption from the intestinal tract. Characteristics of the micelles are dependent on both the lipophilic and hydrophilic properties of these amphiphilic molecules. Vitamin E has a small, weakly hydrophilic head (only one hydroxy per molecule), so that its ability to form micelles in the intestinal lumen requires bile salts and pancreatic excretions. The dispersion-based component of Vitamin E absorption from micelles is equated to the absorption mechanism for fatty acids and fatty acid glycerides. Vitamin E bioavailability depends not only on its dispersion in the intestinal lumen, but also on the co-ingestion of fatty acids and plant sterols, by gene regulating intestinal uptake, by intracellular trafficking, and by lipoprotein secretion of vitamin E; as has been described in the art (Adv. Condensed Matter Physics, 2015, Article ID 151683:22; J Obstet Gynaecol Can, 2009, 31:210-217; Free Radic Res Commun, 1991, 14:229-246; J Clin Invest, 1967, 46:1695-1703).

The ‘dispersion model’ and the low bioavailability of Vitamin E together form the rationale for synthesizing Vitamin E molecules that have more hydrophilic head groups (like uronic acid esters) to improve its absorption by enabling more effective dispersion through micelle formation. Extension of the fatty acid/fatty acid monoglyceride absorption model to include Vitamin E-monoglyceride and more other Vitamin E esters that have more hydrophilic heads, absorption of Vitamin E from the intestinal lumen is thereby improved. By harnessing natural metabolic acids to form these esters, regeneration of Vitamin E upon in vivo hydrolysis will produce only the associated physiological hydrophilic acid, thereby negating concerns for toxic by-products.

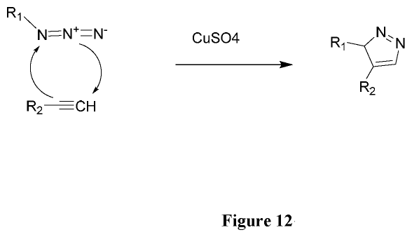

FIG. 12 shows the general reaction to provide for improved bioavailability of Vitamin E analogues. Glyceric acid is first esterified with trifluoroacetic anhydride, then reacted with Vitamin E to form the protected Vitamin E glyceride. This is hydrolyzed with dilute trifluoacetic acid, taken into diethyl ether and the ether fraction is washed with water to afford the desired monoglyceride.

### Example 11: Click Reactions Between Vitamin E Derivatives and Fluorescent Reporter Moieties

The general copper catalyzed click reaction involves alkynes and azides, is depicted in the generic scheme shown in FIG. 12.

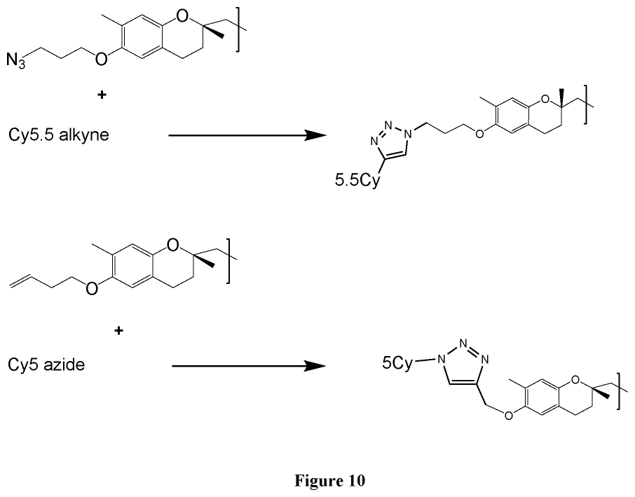

Using standard reaction conditions, azide or alkyne-derivatized Vitamin E, prepared according methods described in this invention, can be reacted with reporting elements via click chemistry. Thus, any functionally-derivatized Vitamin E analogue can be labeled with any of the appropriately decorated commercially available RE (e.g., fluorescent dyes), as depicted in FIG. 10. This procedure can be used to prepare Vitamin E-RE adducts in which the RE is a fluorescent dye, a moiety with a specific chromophore, an NMR-responding moiety, or a moiety with other desired properties.

While particular embodiments of the present invention have been described in the foregoing, it is to be understood that other embodiments are possible within the scope of the invention and are intended to be included herein. It will be clear to any person skilled in the art that modifications of and adjustments to this invention, not shown, are possible without departing from the spirit of the invention as demonstrated through the exemplary embodiments. The invention is therefore to be considered limited solely by the scope of the appended claims.

What is claimed is:

1. A compound of formula (I)
Vitamin E-LNK-REG (I)
wherein Vitamin E is alpha-tocopherol, beta-tocopherol, gamma-tocopherol, delta-tocopherol, alpha-tocotrienol, beta-tocotrienol, gamma-tocotrienol, or delta-tocotrienol;
wherein LNK is a linear or branched hydrocarbon or substituted hydrocarbon linked to the C6 oxygen on the Vitamin E component via an ether, carbamate or ester bond and having a reactive centre suitable for displacement by a nucleophilic reporter element; and
wherein REG is a reporter element or group that is a chemical nucleophile.

2. The compound of claim 1, wherein REG is a halide, azide, reporting moiety that acts as a fluorophore, chromophore, a radioactive element, or a nuclear magnetic resonance responsive center˜.

3. The compound of claim 2 wherein the halide is iodine, bromine, fluorine, or chlorine.

4. The compound of claim 2 wherein the halide is a radioisotope of is Iodine, Bromine, Fluorine, Chlorine.

5. The compound of claim 2 wherein the nuclear magnetic resonance responsive center is a mono-fluorinated, di-fluorinated, tri-fluorinated or polyfluorinated entity.

6. The compound of claim 2 wherein the fluorophore is nitrobenzoxadiazole, or sulfo-cyanine5 fluor.

7. The compound of claim 2 wherein the chromophore is an activated aromatic.

8. The compound of claim 7 wherein the activated aromatic is a nitrated aromatic.

9. A compound

10. A compound of formula (II)
Vitamin E-LNK-GRP (II)
wherein Vitamin E is alpha-tocopherol, beta-tocopherol, gamma-tocopherol, delta-tocopherol, alpha-tocotrienol, beta-tocotrienol, gamma-tocotrienol, or delta-tocotrienol;
wherein LNK is a linear or branched hydrocarbon or substituted hydrocarbon linked to the C6 oxygen on the Vitamin E component via an ether, carbamate or ester bond and having a reactive centre suitable for displacement by a nucleophilic reporter element; and
wherein GRP is a functional element that modifies a property of the Vitamin E component of the compound.

11. The compound of claim 10 wherein GRP is a mono-saccharide, di-saccharide, poly-saccharide, glyceric acid, amino acid, or inorganic acid; resulting in reduction of the lipophilicity of the Vitamin E component of the compound.

12. The compound of claim 10 wherein the mono-saccharide, di-saccharide, or poly-saccharide is uronic acid, gluconic acid, glycuronic acid, ascorbic acid, lacturonic acid, or saccharic acid.

13. The compound of claim 10 wherein GRP provides for improved oral absorption in a mammal.

14. The compound of claim 13 wherein LNK is linked to the C6 oxygen on the Vitamin E component by a di-ester and GRP is a glyceride.

15. The compound of claim 10 wherein GRP provides for improved oral bioavailability in a mammal.

16. The compound of claim 13 wherein LNK is linked to the C6 oxygen on the Vitamin E component by a di-ester and GRP is a glyceride.

17. The compound of claim 10 wherein LNK is linked to the C6 oxygen on the Vitamin E component by mono esterification with glyceric acid or mono-esterification via a dicarboxylate linker, such that the compound may undergo acid hydrolysis to yield Vitamin E, glycerin and dicarboxylate.

18. A method to isolate Vitamin E from natural plant oil distillate containing Vitamin E comprising esterification of hydroxy groups on glucuronic acid sugar by addition of trifluoroacetate generating protected glucuronic acid, addition of the protected glucuronic acid to said natural plant oil distillate generating Vitamin E glucoronate, addition of dilute fluoroacetate to said Vitamin E glucoronate and extracting Vitamin E from the resulting mixture with water.

19. The method of claim 18 wherein extracting Vitamin E from the resulting mixture with water further comprises addition of diethyl ether and dilute aqueous sodium carbonate.

20. A compound of formula (III) 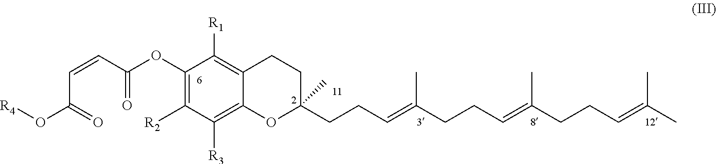
Wherein R₁ is H, or CH₃; R₂ is H or CH₃; R₃ is H or CH₃; and R₄ is H, Vitamin C, or H₂NC(CH₂OH)₃.

21. A compound of formula (IV) 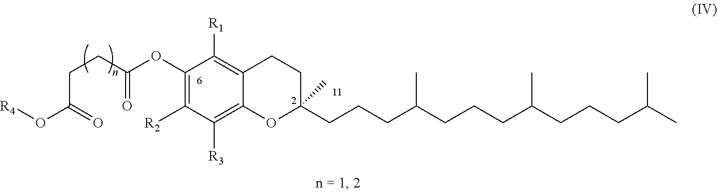
Wherein R₁ is H, or CH₃; R₂ is H or CH₃; R₃ is H or CH₃; and R₄ is H, Vitamin C, or H₂NC(CH₂OH)₃.
